SEQUENCE LISTING (48 sequences)

SEQ ID NO: 1 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 2 - protein, 387 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGSLDRDEVFVPKPKSKVAWFSKYLNNPLGRAVSLLVTLTIGWPMYLAFNVSGRPYDSFASHYHPYAPIYSNRERLLIYVSDVALFSVTYSLYRVATLKGLVWLLCVYGVPLLIVNGFLVTITYLQHTHFALPHYDSSEWDWLKGALATMDRDYGILNKVFHHITDTHVAHHLFSTMPHYHAMEATNAIKPILGEYYQFDDTPFYKALWREARECLYVEPDEGTSEKGVYWYRNKY

SEQ ID NO: 3 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaattcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 4 - protein, 387 aa, Glycine max
MGLAKETTMGGRGRVAKVEVQGKKPLSRVPNTKPPFTVGQFKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGSLDRDEVFVPKPKSKVAWFSKYLNNPLGRAVSLLVTLTIGWPMYLAFNVSGRPYDSFASHYHPYAPIYSNRERLLIYVSDVALFSVTYSLYRVATLKGLVWLLCVYGVPLLIVNGFLVTITYLQHTHFALPHYDSSEWDWLKGALATMDRDYGILNKVFHHITDTHVAHHLFSTMPHYHAMEATNAIKPILGEYYQFDDTPFYKALWREARECLYVEPDEGTSEKGVYWYRNKY

SEQ ID NO: 5 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtggatgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 6 - protein, 387 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWMIAHECGHHAFSKYQWVDDVVGLTLHSTLLVPYFSWKISHRRHHSNTGSLDRDEVFVPKPKSKVAWFSKYLNNPLGRAVSLLVTLTIGWPMYLAFNVSGRPYDSFASHYHPYAPIYSNRERLLIYVSDVALFSVTYSLYRVATLKGLVWLLCVYGVPLLIVNGFLVTITYLQHTHFALPHYDSSEWDWLKGALATMDRDYGILNKVFHHITDTHVAHHLFSTMPHYHAMEATNAIKPILGEYYQFDDTPFYKALWREARECLYVEPDEGTSEKGVYWYRNKY

SEQ ID NO: 7 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggtttccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 8 - protein, 387 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGFLDRDEVFVPKPKSKVAWFSKYLNNPLGRAVSLLVTLTIGWPMYLAFNVSGRPYDSFASHYHPYAPIYSNRERLLIYVSDVALFSVTYSLYRVATLKGLVWLLCVYGVPLLIVNGFLVTITYLQHTHFALPHYDSSEWDWLKGALATMDRDYGILNKVFHHITDTHVAHHLFSTMPHYHAMEATNAIKPILGEYYQFDDTPFYKALWREARECLYVEPDEGTSEKGVYWYRNKY

SEQ ID NO: 9 - DNA, 1164 bp, Glycine max; identical to SEQ ID NO: 5

SEQ ID NO: 10 - protein, 387 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGSLDRDEVFVSKPKSKVAWFSKYLNNPLGRAVSLLVTLTIGWPMYLAFNVSGRPYDSFASHYHPYAPIYSNRERLLIYVSDVALFSVTYSLYRVATLKGLVWLLCVYGVPLLIVNGFLVTITYLQHTHFALPHYDSSEWDWLKGALATMDRDYGILNKVFHHITDTHVAHHLFSTMPHYHAMEATNAIKPILGEYYQFDDTPFYKALWREARECLYVEPDEGTSEKGVYWYRNKY

SEQ ID NO: 11 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtgacctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 12 - protein, 193 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGSLDRDEVFVPKPKSKVAWFSKYLNNPLGRAVSLLVTLTIG

SEQ ID NO: 13 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctgatagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 14 - protein, 387 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGSLDRDEVFVPKPKSKVAWFSKYLNNPLGRAVSLLVTLTIGWPMYLAFNVSDRPYDSFASHYHPYAPIYSNRERLLIYVSDVALFSVTYSLYRVATLKGLVWLLCVYGVPLLIVNGFLVTITYLQHTHFALPHYDSSEWDWLKGALATMDRDYGILNKVFHHITDTHVAHHLFSTMPHYHAMEATNAIKPILGEYYQFDDTPFYKALWREARECLYVEPDEGTSEKGVYWYRNKY

SEQ ID NO: 15 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcttcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 16 - protein, 387 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGSLDRDEVFVPKPKSKVAWFSKYLNNPLGRAVSLLVTLTIGWPMYLAFNVSGRPYDSFASHYHPYAPIYSNRERLLIYVSDVALFSVTYSLYRVATLKGLVWLLCVYGVPLLIVNGFLVTITYLQHTHFALLHYDSSEWDWLKGALATMDRDYGILNKVFHHITDTHVAHHLFSTMPHYHAMEATNAIKPILGEYYQFDDTPFYKALWREARECLYVEPDEGTSEKGVYWYRNKY

SEQ ID NO: 17 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgtctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 18 - protein, 387 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGSLDRDEVFVPKPKSKVAWFSKYLNNPLGRAVSLLVTLTIGWPMYLAFNVSGRPYDSFASHYHPYAPIYSNRERLLIYVSDVALFSVTYSLYRVATLKGLVWLLCVYGVPLLIVNGFLVTITYLQHTHFALSHYDSSEWDWLKGALATMDRDYGILNKVFHHITDTHVAHHLFSTMPHYHAMEATNAIKPILGEYYQFDDTPFYKALWREARECLYVEPDEGTSEKGVYWYRNKY

SEQ ID NO: 19 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaagacactgtggagagaagcgagagagtgcctctatgtggagccagatgaaggaacatccgagaagggcgtgtattggtacaggaacaagtattga

SEQ ID NO: 20 - protein, 387 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGSLDRDEVFVPKPKSKVAWFSKYLNNPLGRAVSLLVTLTIGWPMYLAFNVSGRPYDSFASHYHPYAPIYSNRERLLIYVSDVALFSVTYSLYRVATLKGLVWLLCVYGVPLLIVNGFLVTITYLQHTHFALPHYDSSEWDWLKGALATMDRDYGILNKVFHHITDTHVAHHLFSTMPHYHAMEATNAIKPILGEYYQFDDTPFYKTLWREARECLYVEPDEGTSEKGVYWYRNKY

SEQ ID NO: 21 - DNA, 1164 bp, Glycine max
atgggtctagcaaaggaaacaataatgggaggtggaggccgtgtggccaaagttgaaattcagcagaagaagcctctctcaagggttccaaacacaaagccaccattcactgttggccaactcaagaaagccattccaccgcactgctttcagcgttccctcctcacttcattgtcctatgttgtttatgacctttcattggctttcattttctacattgccaccacctacttccacctcctccctcaccccttttccctcattgcatggccaatctattgggttctccaaggttgcattcttactggcgtgtgggtgattgctcacgagtgtggtcaccatgccttcagcaagtacccatgggttgatgatgttatgggtttgaccgttcactcagcacttttagtcccttatttctcatggaaaataagccatcgccgccaccactccaacacgggttcccttgaccgtgatgaagtgtttgtcccaaaaccaaaatccaaagttgcatggtacaccaagtacctgaacaaccctctaggaagggctgcttctcttctcatcacactcacaatagggtggcctttgtatttagccttcaatgtctctggcagaccctatgatggttttgctagccactaccacccttatgctcccatatattcaaatcgtgagaggcttttgatctatgtctctgatgttgctttgttttctgtgacttacttgctctaccgtgttgcaactatgaaagggttggtttggctgctatgtgtttatggggtgccattgctcattgtgaacggttttcttgtgaccatcacatatctgcagcacacacactatgccttgcctcactatgattcatcagaatgggattggctgaggggtgctttggcaactatggacagagattatggaattctgaacaaggtgtttcaccacataactgatactcatgtggctcaccatcttttctctacaatgccacattaccatgcaacggaggcaaccaatgcaatgaagccaatattgggtgagtactaccgatttgatgacacaccattttacaaggcactgtggagagaagcaagagagtgcctctatgtggagccagatgaaggaacatccgagaagggcgtgtattggtacaggaacaagtattga

SEQ ID NO: 22 - protein, 387 aa, Glycine max
MGLAKETIMGGGGRVAKVEIQQKKPLSRVPNTKPPFTVGQLKKAIPPHCFQRSLLTSLSYVVYDLSLAFIFYIATTYFHLLPHPFSLIAWPIYWVLQGCILTGVWVIAHECGHHAFSKYPWVDDVMGLTVHSALLVPYFSWKISHRRHHSNTGSLDRDEVFVPKPKSKVAWYTKYLNNPLGRAASLLITLTIGWPLYLAFNVSGRPYDGFASHYHPYAPIYSNRERLLIYVSDVALFSVTYLLYRVATMKGLVWLLCVYGVPLLIVNGFLVTITYLQHTHYALPHYDSSEWDWLRGALATMDRDYGILNKVFHHITDTHVAHHLFSTMPHYHATEATNAMKPILGEYYRFDDTPFYKALWREARECLYVEPDEGTSEKGVYWYRNKY

SEQ ID NO: 23 - DNA, 1164 bp, Glycine max
atgggtctagcaaaggaaacaataatgggaggtggaggccgtgtggccaaagttgaaattcagcagaagaagcctctctcaagggttccaaacacaaagccaccattcactgttggccaactcaagaaagccattccaccgcactgctttcagcgttccctcctcacttcattgtcctatgttgtttatgacctttcattggctttcattttctacattgccaccacctacttccacctcctccctcaccccttttccctcattgcatggccaatctattgggttctccaaggttgcattcttactggcgtgtgggtgattgctcacgagtgtggtcaccatgccttcagcaagtacccatgggttgatgatgttatgggtttgaccgttcactcagcacttttagtcccttatttctcatggaaaataagccatcgccgccaccactccaacacgggttcccttgaccgtgatgaagtgtttgtcccaaaaccaaaatccaaagttgcatggtacaccaagtacctgaacaaccctctaggaagggctgcttctcttctcatcacactcacaatagggtggcctttgtatttagccttcaatgtctctggcagaccctatgatggttttgctagccactaccacccttatgctcccatatattcaaatcgtgagaggcttttgatctatgtctctgatgttgctttgttttctgtgacttacttgctctaccgtgttgcaactatgaaagggttggtttggctgctatgtgtttatggggtgccattgctcattgtgaacggttttcttgtgaccatcacatatctgcagcacacacactatgccttgtctcactatgattcatcagaatgggattggctgaggggtgctttggcaactatggacagagattatggaattctgaacaaggtgtttcaccacataactgatactcatgtggctcaccatcttttctctacaatgccacattaccatgcaacggaggcaaccaatgcaatgaagccaatattgggtgagtactaccgatttgatgacacaccattttacaaggcactgtggagagaagcaagagagtgcctctatgtggagccagatgaaggaacatccgagaagggcgtgtattggtacaggaacaagtattga

SEQ ID NO: 24 - protein, 387 aa, Glycine max
MGLAKETIMGGGGRVAKVEIQQKKPLSRVPNTKPPFTVGQLKKAIPPHCFQRSLLTSLSYVVYDLSLAFIFYIATTYFHLLPHPFSLIAWPIYWVLQGCILTGVWVIAHECGHHAFSKYPWVDDVMGLTVHSALLVPYFSWKISHRRHHSNTGSLDRDEVFVPKPKSKVAWYTKYLNNPLGRAASLLITLTIGWPLYLAFNVSGRPYDGFASHYHPYAPIYSNRERLLIYVSDVALFSVTYLLYRVATMKGLVWLLCVYGVPLLIVNGFLVTITYLQHTHYALSHYDSSEWDWLRGALATMDRDYGILNKVFHHITDTHVAHHLFSTMPHYHATEATNAMKPILGEYYRFDDTPFYKALWREARECLYVEPDEGTSEKGVYWYRNKY

SEQ ID NO: 25 - DNA, 22 bp, Artificial Sequence
  primer
tgagggattgtagttctgttgg

SEQ ID NO: 26 - DNA, 20 bp, Artificial Sequence
  primer
agcgtgcattttaggcagaa

SEQ ID NO: 27 - DNA, 20 bp, Artificial Sequence
  primer
tggccaaagtggaagttcaa

SEQ ID NO: 28 - DNA, 24 bp, Artificial Sequence
  primer
attggttgctccatcaatacttgt

SEQ ID NO: 29 - DNA, 22 bp, Artificial Sequence
  primer
tgatgacacaccattttaccag

SEQ ID NO: 30 - DNA, 30 bp, Artificial Sequence
  primer
cattctactaattatgtactaatacatgac

SEQ ID NO: 31 - DNA, 24 bp, Artificial Sequence
  primer
tcccattctgatgaatcgtcctga

SEQ ID NO: 32 - DNA, 22 bp, Artificial Sequence
  primer
tgatgttgctttgttttctgtg

SEQ ID NO: 33 - DNA, 20 bp, Artificial Sequence
  primer
ccatcactccaacacaggat

SEQ ID NO: 34 - DNA, 21 bp, Artificial Sequence
  primer
cataggccaccctattgtgag

SEQ ID NO: 35 - DNA, 25 bp, Artificial Sequence
  primer
ccatgaagcagttgctgaagctgat

SEQ ID NO: 36 - DNA, 28 bp, Artificial Sequence
  primer
cctagagggttgtttaagtacttggaaa

SEQ ID NO: 37 - DNA, 23 bp, Artificial Sequence
  primer
tcagcaacaacaactgaactgaa

SEQ ID NO: 38 - DNA, 22 bp, Artificial Sequence
  primer
tcgctacaagctgtttcacaat

SEQ ID NO: 39 - DNA, 21 bp, Artificial Sequence
  primer
gtggccaaagttgaaattcag

SEQ ID NO: 40 - DNA, 23 bp, Artificial Sequence
  primer
tgcttggttcatcaatacttgtt

SEQ ID NO: 41 - protein, 383 aa, Ricinus communis
MGAGGRMSVPPPSKKVESDDLKRAPSSKPPFTLGQIKKAIPPHCFKRSIPRSFSYVVYDLTIAFLFYYVATNYFHLLPEPLSYVAWPIYWALQGCVLTGVWVIAHECGHHAFSDYQLLDDVVGLILHSCLLVPYFSWKHSHRRHHSNTGSLERDEVFVPKKKSSIRWYSKYLNNPPGRIMTIAVTLTLGWPLYLAFNVSGRPYDRFACHYDPYGPIYNDRERIEIFISDAGVLAVTFGLYQLAIAKGLAWVVCVYGVPLLVVNSFLVLITFLQHTHPALPHYDSSEWDWLRGALATVDRDYGILNKVFHNITDTHVAHHLFSTMPHYHAMEATKAIKPILGEYYQFDGTSFYKAMWREAKECIYVEKDDAEQNGGVFWYNNKF

SEQ ID NO: 42 - protein, 383 aa, Arabidopsis thaliana
MGAGGRMPVPTSSKKSETDTTKRVPCEKPPFSVGDLKKAIPPHCFKRSIPRSFSYLISDIIIASCFYYVATNYFSLLPQPLSYLAWPLYWACQGCVLTGIWVIAHECGHHAFSDYQWLDDTVGLIFHSFLLVPYFSWKYSHRRHHSNTGSLERDEVFVPKQKSAIKWYGKYLNNPLGRIMMLTVQFVLGWPLYLAFNVSGRPYDGFACHFFPNAPIYNDRERLQIYLSDAGILAVCFGLYRYAAAQGMASMICLYGVPLLIVNAFLVLITYLQHTHPSLPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 43 - protein, 39 aa, Glycine max
VTYLHHHGHHQKLPWYRGKEWSYLRGGLTTVDRDYGWIN

SEQ ID NO: 44 - protein, 39 aa, Glycine max; identical to SEQ ID NO: 43

SEQ ID NO: 45 - protein, 39 aa, Glycine max
VTYLHHHGYKQKLPWYRGQEWSYLRGGLTTVDRDYGWIN

SEQ ID NO: 46 - protein, 37 aa, Glycine max
ITYLQHTHYALPHYDSSEWDWLRGALATMDRDYGILN

SEQ ID NO: 47 - protein, 37 aa, Glycine max
ITYLQHTHFALPHYDSSEWDWLKGALATMDRDYGILN

SEQ ID NO: 48 - protein, 37 aa, Arabidopsis thaliana
ITYLQHTHPSLPHYDSSEWDWLRGALATVDRDYGILN

(19) United States
(12) Patent Application Publication
(10) Pub. No.: US 2017/0283820 A1
(43) Pub. Date: Oct. 5, 2017

(54) HIGH OLEIC ACID SOYBEAN SEEDS

(71) Applicant: The United States of America, as represented by the Secretary of Agriculture, Washington, DC (US)

(72) Inventor: Karen A Hudson, West Lafayette, IN (US)

(21) Appl. No.: 15/089,689

(22) Filed: Apr. 4, 2016

(51) Int. Cl. C12N 15/82; C12Q 1/68

(52) U.S. Cl. CPC C12N 15/8247; C12Q 1/6895; C12Q 2600/13; C12Q 2600/156

(57) ABSTRACT

A genetically altered soybean plant, its parts (including seeds, cells, flowers, pollen), and its progeny produces at least one altered delta-twelve fatty acid desaturase 2 enzyme (FAD2), namely an altered FAD2-1A, an altered FAD2-1B, or both. This genetically altered soybean plant has reduced FAD2 enzymatic activity in its seeds, thereby producing higher amounts of oleic acid in its seeds than a wild-type soybean plant produces. Methods of generating this genetically altered soybean plant are provided.

### BACKGROUND OF THE INVENTION

### Field of the Invention

This invention relates to a genetically altered soybean plant and its seeds that produce higher amounts of oleic acid in the seeds compared to the amount of oleic acid produced in the seeds of wild-type soybean plants. This invention also relates to the mutations that result in this phenotype.

### Description of Related Art

Soybean oil accounted for 63% of vegetable oil consumption in the United States, and 28% worldwide. Oleic acid is a fatty acid found in soybean (Glycine max) seed oil that is most desirable because of several benefits: heat-stability, health and versatility. Typically, soybean oil's fatty acid composition is 13% palmitic acid (16:0), 4% stearic acid (18:0), 20% oleic acid (18:1), 55% linoleic acid (18:2), and 8% linolenic acid (18:3). In the lipid biosynthetic pathway, conversion of oleic acid (18:1) precursors to linoleic acid (18:2) precursors is catalyzed by the delta-twelve fatty acid desaturase 2 enzyme (FAD2). Efforts have been made to increase the levels of oleic acid in soybean seeds. A variety of genetic and biotechnological approaches to increase the oleic acid levels in soybean seeds have been tried. See, e.g., Clemente and Cahoon, 2009, Plant Physiol. 151:1030-1040. Genetic approaches to reduce levels of linolenic acid naturally occurring in soybeans are preferred to the hydrogenation process that can introduce trans-fats (Fehr, W. R., 2007, Crop Sci. 47:S72-S87). Recently, soybean lines that produce higher proportions of oleic acid in the seeds have been generated and are being grown in commercial production.

Oleic acid is produced in the cytoplasm by the desaturation of stearic acid, and is typically further desaturated to produce linoleic acid by the action of the microsomal omega-6 fatty acid desaturase (FAD) enzymes which are encoded by the FAD genes (Ohlrogge and Browse, 1995, Plant Cell 7:957-970). Soybean has five characterized FAD2 genes. FAD2-1A and FAD2-1B (Glyma.10G278000 and Glyma.20G111000, respectively) are 94% identical at the amino acid level and are expressed at high levels in developing soybean seeds (Anai, et al., 2008, Breeding Sci. 58:447-452). FAD2-2B and FAD2-2C are expressed in soybean leaves and seeds, and FAD2-2A is not expressed in the soybean Williams-82 cultivar (Heppard, et al., 1996, Plant Physiol. 110:311-319; Schlueter, et al., 2007, Crop Sci.:S14-S26).

Of these five genes, FAD2-1A and FAD2-1B have the largest effect on oleic acid biosynthesis in soybean seed tissue. As such, they are the targets for breeders seeking to increase oleic acid levels in soybean seeds. The soybean line M23 contains a deletion of the FAD2-1A open reading frame, and line KK21 carries a single nucleotide deletion resulting in a frameshift in the FAD2-1A transcript and result in seeds with approximately 48% oleic acid of the total fatty acids in the seeds (Anai, et al., 2008). Plant introduction 603452 carries a single nucleotide deletion in the FAD2-1A coding region which causes a frameshift and early termination after 191 amino acids and results in seeds with ˜35% oleic acid out of the total fatty acids in the seeds (Pham, et al., 2011, Theor. Appl. Genet. 123:793-802. Point mutations in FAD2-1A identified by reverse genetics approaches (a missense mutation that converts the serine at position 117 to asparagine) result in 35% oleic acid of the total fatty acids in the seeds (Dierking and Bilyeu, 2009, BMC Plant Bio. 9:89). Turning to FAD2-1B mutants, two SNPs identified by reverse genetics in FAD2-1B result in a significant elevation in oleic acid levels alone (Hoshino, et al., 2010, Breeding Sci. 60:419-425).

Furthermore, the combination of deleterious mutations in FAD2-1A and FAD2-1B can result in a synergistic increase in the oleic acid levels in seeds, typically increasing oleic acid levels to 80%-90% of the total fatty acids. See, e.g., Hoshino, et al., 2010; Pham, et al., 2010, BMC Plant Bio. 10:195; and Pham, et al., 2011. These oleic acid levels approach the oleic acid levels in soybean seeds achieved using molecular biology techniques that reduce enzyme levels of multiple FAD genes. For example, TALEN (transcription activator-like effector nuclease) was used to simultaneously introduce deletion mutations in both FAD2-1A and FAD2-1B, resulting in a double mutant soybean plant with 80% oleic acid levels of the total fatty acids in the seeds (Haun, et al., 2014, Plant Biotech. J. 12:934-940). RNAi targeting FAD2-1 achieved soybean seeds having oleic acid levels ranging from 60% to 80% of the total fatty acids in the seeds. See, Buhr, et al., 2002, Plant J. 30:155-163; Mroczka, et al., 2010, Plant Physiol. 153:882-891; and Wagner, et al., 2011, Plant Biotech. J. 9:723-728).

However, a need still exists for genetically altered soybean plants containing mutations in FAD2-1A and/or FAD2-1B to achieve oleic acid levels of approximately 80% or higher of the total fatty acids in the seeds. In particular, a need also exists for soybean plants with mutations that are not based on transgenic modification that could be used to develop non-transgenic high oleic soybean varieties.

### BRIEF SUMMARY OF THE INVENTION

It is an object of this invention to have a genetically altered soybean plant and parts thereof that contain an altered delta-twelve fatty acid desaturase 2-1B enzyme (FAD2-1B) and an altered delta-twelve fatty acid desaturase 2-1A enzyme (FAD2-1A), such that the altered FAD2-1A and FAD2-1B have reduced enzymatic activity compared to the enzymatic activity of wild-type FAD2-1A and FAD2-1B, respectively. It is a further object of this invention that this genetically altered soybean plant is capable of producing seeds containing higher amounts of oleic acid compared to the amount of oleic acid in the seeds of a wild-type soybean plant having wild-type FAD2-1B and wild-type FAD2-1A. It is another object of this invention to have seeds, a cell, protoplast containing cells, a germplasm, pollen, leaves, ovaries, etc., of this genetically altered soybean plant. It is another object of this invention that the fatty acid content of the seeds of this genetically altered soybean plant contains approximately 70%, 71%, 72%, 73%, 74%, 75%, 76%, 77%, 78%, 79%, 80%, 81%, 82%, or higher oleic acid.

It is another object of this invention to have a genetically altered soybean plant having a P284S mutation in FAD2-1B and a FAD2-1A mutation that can be FAD2-1A_{L41F}, FAD2-1A_{V106M}, FAD2-1A_{S154F}, FAD2-1A_{P163S}, FAD2-1A_{W194STOP}, FAD2-1A_{G204D}, FAD2-1A_{P284L}, FAD2-1A_{P284S}, or FAD2-1A_{A358T}. It is another object of this invention to have seeds, a cell, protoplast containing cells, a germplasm, pollen, leaves, ovaries, etc., of this genetically altered soybean plant. It is another object of this invention that the fatty acid content of the seeds of this genetically altered soybean plant contains approximately 70%, 71%, 72%, 73%, 74%, 75%, 76%, 77%, 78%, 79%, 80%, 81%, 82%, or higher oleic acid.

It is an object of this invention to have a genetically altered soybean plant having a P284S mutation in FAD2-1B and a W194STOP mutation in FAD2-1A. It is another object of this invention that this genetically altered soybean plant was deposited with ATCC and has accession number PTA-122890. It is another object of this invention to have seeds, a cell, protoplast containing cells, a germplasm, pollen, leaves, ovaries, etc., of this genetically altered soybean plant. It is another object of this invention that the fatty acid content of the seeds of this genetically altered soybean plant contains approximately 70%, 71%, 72%, 73%, 74%, 75%, 76%, 77%, 78%, 79%, 80%, 81%, 82%, or higher oleic acid.

It is an object of this invention to have a method for constructing a genetically altered soybean plant having an altered delta-twelve fatty acid desaturase 2-1B enzyme (FAD2-1B) and an altered delta twelve fatty acid desaturase 2-1A enzyme (FAD2-1A), the altered soybean plant being capable of producing seeds with a fatty acid content of at least 70% oleic acid. It is another object of this invention that the method have the steps of introducing a nucleic acid encoding an altered FAD2-1B enzyme having a P284S mutation into a wild-type soybean plant to provide a first genetically altered soybean plant; selecting a first genetically altered soybean plant that is homozygous for this altered FAD2-1B enzyme; introducing a nucleic acid encoding the altered FAD2-1A enzyme into the first genetically altered soybean plant to provide a second genetically altered soybean plant, such that the altered FAD2-1A enzyme can be FAD2-1A_{L41F}, FAD2-1A_{V106M}, FAD2-1A_{S154F}, FAD2-1A_{P163S}, FAD2-1A_{W194STOP}, FAD2-1A_{G204D}, FAD2-1A_{P284L}, FAD2-1A_{P284S}, or FAD2-1A_{A358T}; and selecting the second genetically altered soybean plant that is homozygous for the altered FAD2-1B enzyme and altered FAD2-1A enzyme; thereby constructing the genetically altered soybean plant capable of producing seeds with a fatty acid content of at least 70% oleic acid. It is a further object of this invention that the introducing of at least one nucleic acid encoding an altered FAD2-1A or FAD2-1B occurs via introgression, genomic editing, or exposing the wild-type soybean plant to a mutagen, and that selecting the genetically altered soybean plant occurs via marker assisted selection. It is another object of this invention to have an altered soybean plant and parts thereof (including seeds) made using this method.

It is an object of this invention to have a method for constructing a genetically altered soybean plant having an altered delta-twelve fatty acid desaturase 2-1B enzyme (FAD2-1B) and an altered delta twelve fatty acid desaturase 2-1A enzyme (FAD2-1A), the altered soybean plant being capable of producing seeds with a fatty acid content of at least 70% oleic acid. It is another object of this invention that the method have the steps of introducing a nucleic acid encoding the altered FAD2-1A enzyme into a wild-type soybean plant to provide a first genetically altered soybean plant, such that the altered FAD2-1A enzyme can be FAD2-1A_{L41F}, FAD2-1A_{V106M}, FAD2-1A_{S154F}, FAD2-1A_{P163S}, FAD2-1A_{W194STOP}, FAD2-1A_{G204D}, FAD2-1A_{P284L}, FAD2-1A_{P284S}, or FAD2-1A_{A358T}; selecting the first genetically altered soybean plant that is homozygous for the altered FAD2-1A enzyme; introducing a nucleic acid encoding an altered FAD2-1B enzyme having a P284S mutation into said first genetically altered soybean plant to provide a second genetically altered soybean plant; and selecting a second genetically altered soybean plant that is homozygous for the altered FAD2-1B enzyme and altered FAD2-1A; thereby constructing the genetically altered soybean plant capable of producing seeds with a fatty acid content of at least 70% oleic acid. It is a further object of this invention that the introducing of at least one nucleic acid encoding an altered FAD2-1A or FAD2-1B occurs via introgression, genomic editing, or exposing the wild-type soybean plant to a mutagen, and that selecting the genetically altered soybean plant occurs via marker assisted selection. It is another object of this invention to have an altered soybean plant and parts thereof (including seeds) made using this method.

Another object of this invention is to have a kit useful for the identification of SNPs in FAD2-1A or FAD2-1B genes of a soybean plant containing a plurality of polynucleotides, optionally a restriction endonuclease, optionally a polymerase, optionally an identifying dye, and optionally instructions, such that the plurality of polynucleotides can be SEQ ID NOs: 29 and 30, SEQ ID NOs: 31 and 32, SEQ ID NOs: 25 and 28, SEQ ID NOs: 27 and 28, SEQ ID NOs: 33 and 34, SEQ ID NOs: 35 and 36, SEQ ID NOs: 37 and 40, or a combination thereof. It is a further object of this invention that optional endonuclease in the kit can be is BstNI when polynucleotides having SEQ ID NOs: 29 and 30 are present; Bsu36I when polynucleotides having SEQ ID NOs: 31 and 32 are present; BsmAI and/or BstEII when polynucleotides having SEQ ID NOs: 25 and 28 are present; Accl and/or Fokl when polynucleotides having SEQ ID NOs: 27 and 28 are present; BamHI when polynucleotides having SEQ ID NOs: 33 and 34 are present; BpuEI when polynucleotides having SEQ ID NOs: 35 and 36 are present; and BsmAI when polynucleotides having SEQ ID NOs: 37 and 40 are present. It is yet another object of this invention to use this kit to determine if a soybean plant contains one or more specific SNPs.

### BRIEF DESCRIPTION OF THE SEVERAL VIEWS OF THE DRAWINGS

FIG. 1 shows the location of the nine different amino acid mutations at conserved residues in FAD2-1A. All nine genetically altered soybean lines carry single-base changes in the FAD2-1A gene that result in either early termination of the protein, or an amino acid change in a conserved residue. CLUSTAL-W multiple sequence alignment shows FAD2 protein sequences from Arabidopsis thaliana (At3g12120) (SEQ ID NO: 42), castor bean (Rco 29613.t000001) (SEQ ID NO: 41), and two members of the soybean FAD2 gene family (FAD2-1A: Glyma10g42470/Glyma.10G278000 (SEQ ID NO: 2), FAD2-1B: Glyma20g24530/Glyma.20G111000 (SEQ ID NO: 22)). In FIG. 1, mutation 1 is FAD2-1A_{L41F}; mutation 2 is FAD2-1A_{V106M}; mutation 3 is FAD2-1A_{S154F}; mutation 4 is FAD2-1A_{P163S}; mutation 5 is FAD2-1A_{W194STOP}; mutation 6 is FAD2-1A_{G204D}; mutation 7 are FAD2-1A_{P284L} and FAD2-1A_{P284S}; mutation 8 is FAD2-1A_{A358T}.

FIG. 2A through FIG. 2H show fatty acid composition of seeds from wild-type, heterozygotes, genetically altered soybean plants for each of the eight genetically altered FAD2-1A soybean lines, demonstrating that the genetic alternations cosegregate with elevated oleic acid phenotype. FIG. 2A shows FAD2-1A_{L41F} line. FIG. 2B shows FAD2-1A_{V106M} line. FIG. 2C shows FAD2-1A_{P163S} line. FIG. 2D shows FAD2-1A_{W194 STOP} line. FIG. 2E shows FAD2-1A_{G204D} line. FIG. 2F shows FAD2-1A_{P284L} line. FIG. 2G shows FAD2-1A_{P284S} line. FIG. 2H shows FAD2-1A_{A358T} line.

FIG. 3 illustrates that the FAD2-1B_{P284S} mutation occurs at a conserved position. Sequence alignment of between 36 and 38 amino acids encoded by six genes show the conservation of the proline at position 284 across FAD2 and FAD3 genes. Shaded residues have >75% identity across the listed fatty acid desaturase enzyme sequences. GmFAD2-1A: Glyma.10g278000 (SEQ ID NO: 47), GmFAD2-1B: Glyma.20g111000 (SEQ ID NO: 46), GmFAD3A: Glyma.14g194300 (SEQ ID NO: 43), GmFAD3B: Glyma.02g227200 (SEQ ID NO: 44), GmFAD3C: Glyma.18g062000 (SEQ ID NO: 45), and A. thaliana FAD2: At3g12120 (SEQ ID NO: 48).

FIG. 4 illustrates the cosegregation for FAD2-1B with elevated oleic phenotype by showing the oleic acid content in segregating F2 seeds genotyped for the P284S polymorphism.

FIG. 5A though FIG. 5D show the fatty acid composition values for four populations of segregating F2 phenotyped and genotyped for FAD2-1A and FAD2-1B. FIG. 5A is the fatty acid composition for FAD2-1A_{P163S}×FAD2-1B_{P284S}. FIG. 5B is the fatty acid composition for FAD2-1A_{V106M}×FAD2-1B_{P284S}. FIG. 5C is the fatty acid composition for FAD2-1A_{L41F}×FAD2-1B_{P284S}. FIG. 5D is the fatty acid composition for FAD2-1B_{P284S}×FAD2-1A_{W194STOP}.

### STATEMENT REGARDING DEPOSIT OF BIOLOGICAL MATERIAL UNDER THE TERMS OF THE BUDAPEST TREATY

On or before Feb. 24, 2016, the inventor deposited 2,500 seeds of soybean seed 14473.1X14184.15-1749/50 (which contains the FAD2-1BP248S and FAD2-1A_{W194STOP} alternations), as described herein, with American Type Culture Collection (ATCC) located at 10801 University Blvd., Manassas, Va. 20110, in a manner affording permanence of the deposit and ready accessibility thereto by the public if a patent is granted. The deposit has been made under the terms of the Budapest Treaty on the International Recognition of the Deposit of Microorganisms for the Purposes of Patent Procedure and the regulations thereunder. The deposit's accession number is ATCC Accession Number PTA-122890.

All restrictions on the availability to the public of soybean seed 14473.1X14184.15-1749/50 (PTA-122890), which has been deposited as described herein will be irrevocably removed upon the granting of a patent covering this particular biological material.

Soybean seed 14473.1X14184.15-1749/50 (PTA-122890) has been deposited under conditions such that access to the organism is available during the pendency of the patent application to one determined by the Commissioner to be entitled thereto under 37 C.F.R. §1.14 and 35 U.S.C §122.

The deposited biological material will be maintained with all the care necessary to keep it viable and uncontaminated for a period of at least five years after the most recent request for the furnishing of a sample of the deposited microorganism, and in any case, for a period of at least thirty (30) years after the date of deposit for the enforceable life of the patent, whichever period is longer.

I, the inventor, for the invention described in this patent application, hereby declare further that all statements regarding this Deposit of the Biological Material made on information and belief are believed to be true and that all statements made on information and belief are believed to be true, and further that these statements are made with knowledge that willful false statements and the like so made are punishable by fine or imprisonment, or both, under section 1001 of Title 18 of the United States Code and that such willful false statements may jeopardize the validity of the instant patent application or any patent issuing thereon.

### DETAILED DESCRIPTION OF THE INVENTION

A need exists for soybean plants that can produce high levels of oleic acid in the seeds. Exposing wild-type soybean seeds (Williams-82 cultivar) with N-nitroso-N-methylurea, a mutagen, generates random genetic variation. Screening the progeny of the exposed seeds for plants that produce higher levels of oleic acid in their seeds compared to the level of oleic acid in the wild-type soybean seeds is one method of identifying these mutant individuals. As discussed and shown below, soybean plants having genetic alterations in fad2-1A and fad2-1B produced higher amounts of oleic acid in their seeds than the amount produced in wild-type soybean seeds. The individual amino acid changes as a result of the DNA changes in FAD2-1A are FAD2-1A_{L41F}; FAD2-1A_{V106M}, FAD2-1A_{S154F}, FAD2-1A_{P163S}, FAD2-1A_{W194STOP}, FAD2-1A_{G204D}, FAD2-1A_{P284L}, FAD2-1A_{P284S}, and FAD2-1A_{A358T}. For FAD2-1B, only one amino acid change was identified, namely FAD2-1B_{P284S}. Next, genetically altered soybean plants containing any one of these altered fad2-1A genes and the altered fad2-1B gene are generated, and these genetically altered soybean plants (double mutants) produced even higher percentage of oleic acid of the total fatty acids in the seeds than the parent plants. So, one embodiment of this invention is a genetically altered soybean plant having the phenotype of any one of these FAD2-1A or FAD2-1B alterations. Another embodiment of this invention is a genetically altered soybean plant having any one of these FAD2-1A alterations and the FAD2-1B alteration. See Table 1 for a list of the wild-type and genetic alterations for FAD2-1A and FAD2-1B, and the respective sequence identification numbers (SEQ ID NOs).

TABLE 1

| | DNA sequence | amino acid sequence |
|---|---|---|
| FAD2-1A (wild-type) | SEQ ID NO: 1 | SEQ ID NO: 2 |
| FAD2-1A_{L41F} | SEQ ID NO: 3 | SEQ ID NO: 4 |
| FAD2-1A_{V106M} | SEQ ID NO: 5 | SEQ ID NO: 6 |
| FAD2-1A_{S154F} | SEQ ID NO: 7 | SEQ ID NO: 8 |
| FAD2-1A_{P163S} | SEQ ID NO: 9 | SEQ ID NO: 10 |
| FAD2-1A_{W194STOP} | SEQ ID NO: 11 | SEQ ID NO: 12 |
| FAD2-1A_{G204D} | SEQ ID NO: 13 | SEQ ID NO: 14 |
| FAD2-1A_{P284L} | SEQ ID NO: 15 | SEQ ID NO: 16 |
| FAD2-1A_{P284S} | SEQ ID NO: 17 | SEQ ID NO: 18 |
| FAD2-1A_{A358T} | SEQ ID NO: 19 | SEQ ID NO: 20 |
| FAD2-1B (wild-type) | SEQ ID NO: 21 | SEQ ID NO: 22 |
| FAD2-1B_{P284S} | SEQ ID NO: 23 | SEQ ID NO: 24 |

In one embodiment of this invention, a genetically altered soybean having one of the described FAD2-1A alterations and the FAD2-1B alteration (see Table 1 supra) will produce seeds containing an amount of oleic acid that is at least 70% of the total fatty acids in the seeds. In other embodiment, these genetically altered soybean plants will produce seeds containing oleic acid that is 73%, 74%, 75%, 76%, 77%, 78%, 79%, 80%, 81%, 82%, or higher of the total fatty acids content in the seeds. For the purposes of this invention, the “percentage” or “amount” of oleic acid in the seed refers to the percentage of oleic acid, on average, present in the seed of the genetically altered soy bean plants described herein based on the total amount of fatty acids present in those seeds. For example, a plant producing “80% oleic acid” means that the amount of oleic acid in the seeds are, on average, equal to 80% of the total fatty acid content of the seeds.

Unless otherwise indicated, a particular nucleic acid sequence for each amino acid substitution (alteration) also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98(1994)). Because of the degeneracy of nucleic acid codons, one can use various different polynucleotides to encode identical polypeptides. Table 2, infra, contains information about which nucleic acid codons encode which amino acids and is useful for determining the possible nucleotide substitutions that are included in this invention.

TABLE 2

| Amino | | Amino | |
|---|---|---|---|
| acid | Nucleic acid codons | acid | Nucleic acid codons |
| Ala/A | GCT, GCC, GCA, GCG | Leu/L | TTA, TTG, CTT, CTC, |
| | | | CTA, CTG |
| Arg/R | CGT, CGC, CGA, CGG, | Lys/K | AAA, AAG |
| | AGA, AGG | | |
| Asn/N | AAT, AAC | Met/M | ATG |
| Asp/D | GAT, GAC | Phe/F | TTT, TTC |
| Cys/C | TGT, TGC | Pro/P | CCT, CCC, CCA, CCG |
| Gln/Q | CAA, CAG | Ser/S | TCT, TCC, TCA, TCG, |
| | | | AGT, AGC |
| Glu/E | GAA, GAG | Thr/T | ACT, ACC, ACA, ACG |
| Gly/G | GGT, GGC, GGA, GGG | Trp/W | TGG |
| His/H | CAT, CAC | Tyr/Y | TAT, TAC |
| Ile/I | ATT, ATC, ATA | Val/V | GTT, GTC, GTA, GTG |
| Stop | TAA, TGA, TAG | | |

The term “plant” includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The class of plants that can be used in the method of the invention is generally as broad as the class of higher and lower plants amenable to the molecular biology and plant breeding techniques described herein, specifically angiosperms (monocotyledonous (monocots) and dicotyledonous (dicots) plants). It includes plants of a variety of ploidy levels, including aneuploid, polyploid, diploid, haploid and hemizygous. The genetically altered plants described herein soybean plants.

The term “nucleic acid” as used herein, refers to a polymer of ribonucleotides or deoxyribonucleotides. Typically, “nucleic acid” polymers occur in either single- or double-stranded form, but are also known to form structures comprising three or more strands. The term “nucleic acid” includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Exemplary analogs include, without limitation, phosphorothioates, phosphoramidates, methyl phosphonates, chiral-methyl phosphonates, 2-O-methyl ribonucleotides, peptide-nucleic acids (PNAs). “DNA”, “RNA”, “polynucleotides”, “polynucleotide sequence”, “oligonucleotide”, “nucleotide”, “nucleic acid”, “nucleic acid molecule”, “nucleic acid sequence”, “nucleic acid fragment”, and “isolated nucleic acid fragment” are used interchangeably herein.

The term “label” as used herein, refers to a composition detectable by spectroscopic, photochemical, biochemical, immunochemical, or chemical means. Exemplary labels include ³²P, fluorescent dyes, electron-dense reagents, enzymes (e.g., as commonly used in an ELISA), biotin, digoxigenin, or haptens and proteins for which antisera or monoclonal antibodies are available.

As used herein a nucleic acid “probe”, oligonucleotide “probe”, or simply a “probe” refers to a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (e.g., 7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. In one embodiment, probes are directly labeled as with isotopes, chromophores, lumiphores, chromogens, etc. In another embodiment probes are indirectly labeled e.g., with biotin to which a streptavidin complex may later bind. By assaying for the presence or absence of the probe, one can detect the presence or absence of the select sequence or subsequence. Thus, a probe is set of polynucleotides that can bind, either covalently, through a linker or a chemical bond, or non-covalently, through ionic, van der Waals, electrostatic, or hydrogen bonds, to a label such that the presence of the probe may be detected by detecting the presence of the label bound to the probe.

The term “primer” as used herein, refers to short nucleic acids, typically a DNA oligonucleotide of at least about 15 nucleotides in length. In one embodiment, primers are annealed to a complementary target DNA strand by nucleic acid hybridization to form a hybrid between the primer and the target DNA strand. Annealed primers are then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

PCR primer pairs are typically derived from a known sequence, for example, by using computer programs intended for that purpose such as Primer (Version 0.5©1991, Whitehead Institute for Biomedical Research, Cambridge, Mass.). One of ordinary skill in the art will appreciate that the specificity of a particular probe or primer increases with its length. Thus, for example, a primer comprising 20 consecutive nucleotides of sequence will anneal to a particular sequence with a higher specificity than a corresponding primer of only 15 nucleotides. Thus, in one embodiment, greater specificity of a nucleic acid primer or probe is attained with probes and primers selected to comprise 20, 25, 30, 35, 40, 50 or more consecutive nucleotides of a selected sequence.

A genetically altered organism is any organism with any changes to its genetic material, whether in the nucleus or cytoplasm (organelle). As such, a genetically altered organism can be a recombinant or transformed organism. A genetically altered organism can also be an organism that was subjected to one or more mutagens or the progeny of an organism that was subjected to one or more mutagens and has mutations in its DNA caused by the one or more mutagens, as compared to the wild-type organism (i.e, organism not subjected to the mutagens). Also, an organism that has been bred to incorporate a mutation into its genetic material is a genetically altered organism. For the purposes of this invention, the organism is a plant.

Once a genetically altered plant has been generated, one can breed it with a wild-type plant and screen for heterozygous F1 generation plants containing the genetic change present in the parent genetically altered plant. Then F2 generation plants can be generated which are homozygous for the mutation. These heterozygous F1 generation plants and homozygous F2 plants, progeny of the original genetically altered plant, are considered genetically altered plants, having the altered genomic material from a parent plant that has been genetically altered.

As discussed briefly above, one can subject a plant's seeds to a mutagen, then grow the seeds, and screen the plants for altered phenotypes. The plants with altered phenotypes will have one or more mutations within the plant's DNA (either within the organelles or nucleus) that cause the altered phenotype. Such genetically altered plants can then be bred as described above to generate homozygous genetically altered plants.

Another way to create mutations in a plant cell is through genomic editing. Recombinant DNA restriction enzymes can be engineered by fusing a nuclease, for example Fokl, with a structure that binds to a site in the plant cell's DNA, as specified by zinc finger, TALEN (transcription activator-like effector nuclease), or by CRISPR (clustered regularly interspaced short palindromic repeat)-Cas9 system, to make a double strand cut within the DNA and replace the excised DNA with an engineered nucleic acids identified from the genetically altered plants described herein. Fokl is a bacterial type IIS restriction endonuclease consisting of an N-terminal DNA-binding domain, which can be made to bind specific DNA sequences in genome and a non-specific DNA cleavage domain at the C-terminal. See, Belhaj, et al., Plant Methods 9(1):39 (2013); Nekrasov, et al., Nat. Biotechnol. 31:691-693 (2013); Voytas, D. F., Annu. Rev. Plant Biol. 64:327-350 (2013); Shan, et al., Nat. Biotech. 31:686-688 (2013); and Li, et al., Methods 69(1):9-16 (2014).

Genetically altered plants having mutations in FAD2-1A and FAD2-1B can be selected using marker assisted selection. Marker-assisted selection is a method of selecting desirable individuals in a breeding scheme based on DNA molecular marker patterns instead of, or in addition to, their phenotypic traits. Marker-assisted selection provides a useful tool that allows for efficient selection of desirable crop traits and is well known in the art (see, e.g., Podlich, et al., Crop Sci. 44:1560-1571 (2004); Ribaut and Hoisington, Trends in Plant Science 3:236-238 (1998); Knapp, S., Crop Science 38:1164-1174 (1998); Hospital, F., Marker-assisted breeding, pp 30-59, in Plant molecular breeding, H. J. Newbury (ed.), Blackwell Publishing and CRC Press (Oxford and Boca Raton).

As is well known in the art, breeders typically improve crops by crossing plants with desired traits, such as high yield or disease resistance, and selecting the best offspring over multiple generations of testing. Thus, new varieties can easily take eight to ten years to develop. In contrast to conventional selection methods, with marker-assisted selection plants are selected based on molecular marker patterns known to be associated with the traits of interest. Thus, marker-assisted selection involves selecting individuals based on their marker pattern (genotype) rather than their observable traits (phenotype). Thus, molecular marker technology offers the possibility to speed up the selection process and thus offers the potential to develop new cultivars quickly.

Therefore, in an exemplary embodiment, marker assisted selection is used to develop new soybean plants having the FAD2-1A and FAD2-1B mutations described herein and thus produce higher amounts of oleic acid in the soybean seeds compared to the amount of oleic acid produced by wild-type soybean seeds (percentage of oleic acid compared to the total amount of fatty acids present in the seeds). In this embodiment, the single nucleotide polymorphisms disclosed herein are used as markers to select for the FAD2-1A and/or FAD2-1B mutations described herein.

In general, the basic procedure for conducting marker assisted selection with DNA markers is as follows: First, extracting DNA from tissue of each individual or family in a population. Second, screening DNA samples via PCR for the molecular marker (SSR, SNP, SCAR, etc.) linked to the trait of interest. Third, separating and scoring PCR products, using an appropriate separation and detection technique. Fourth, identifying individual plants exhibiting or having the desired marker allele. Fifth, combining the marker results with other selection criteria (e.g., phenotypic data or other marker results). Six, selecting the fraction of the population besting meeting the selection criteria, and advancing those selected plant in the breeding program.

The terms “identical” or percent “identity”, in the context of two or more nucleic acids or polypeptide sequences, refer to two or more sequences or subsequences that are the same or have a specified percentage of amino acid residues or nucleotides that are the same (e.g., 80%, 85% identity, 90% identity, 99%, or 100% identity), when compared and aligned for maximum correspondence over a designated region as measured using a sequence comparison algorithm or by manual alignment and visual inspection.

The phrase “high percent identical” or “high percent identity”, in the context of two polynucleotides or polypeptides, refers to two or more sequences or subsequences that have at least about 80%, identity, at least about 81%, 82%, 83%, 84%, 85%, 86%, 87%, 88%, 89%, 90%, 91%, 92%, 93%, 94%, 95%, 96%, 97%, 98%, 99%, or 100% nucleotide or amino acid residue identity, when compared and aligned for maximum correspondence, as measured using a sequence comparison algorithm or by visual inspection. In an exemplary embodiment, a high percent identity exists over a region of the sequences that is at least about 50 residues in length. In another exemplary embodiment, a high percent identity exists over a region of the sequences that is at least about 100 residues in length. In still another exemplary embodiment, a high percent identity exists over a region of the sequences that is at least about 150 residues or more in length. In one exemplary embodiment, the sequences are high percent identical over the entire length of the nucleic acid or protein sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al. (eds.), Current Protocols in Molecular Biology, 1995 supplement).

This invention includes kits that contain at least one pair of polynucleotides which can be used to determine if a plant carries a wild-type or mutated FAD2-1A and/or FAD2-1B genes where the mutation is a single nucleotide change, optionally a restriction endonuclease for identifying the SNP, optionally a polymerase, optionally an identifying dye, and optionally instructions for use of the at least one pair of polynucleotides. One pair of polynucleotides useful for this assay are SEQ ID NO: 25 and SEQ ID NO: 28, which amplify the sequence flanking the polymorphism in the FAD2-1A_{W194STOP} (SEQ ID NO: 11) gene sequence that results in a mutated FAD2-1A that results in increased amount of oleic acid in the genetically altered plant's seed. The amplified PCR product (1259 base pairs) is subjected to digestion by the BstEII restriction enzyme which cleaves only DNA carrying the mutated sequence, and can thus be used to discriminate wild-type plants from heterozygous or homozygous mutant plants. Another pair of polynucleotides useful for this assay are SEQ ID NO: 37 and SEQ ID NO: 40, where SEQ ID NO: 37 and SEQ ID NO: 40 are PCR primers flanking the DNA sequence around the polymorphism in FAD2-1B (SEQ ID NO: 23) that results in an mutated FAD2-1B that results in increased amount of oleic acid in the genetically altered plant's seed. The amplified PCR product can be subjected to digestion with the restriction enzyme BsmAI which cleaves only the PCR product from the mutant allele, and can thus be used to distinguish wild-type plants from heterozygous or homozygous mutant plants. During crossing one genetically altered plant expressing the FAD2-1A/FAD2-1B phenotype with a non-genetically altered plant into which one wants to breed and express the FAD2-1A/FAD2-1B phenotype, one can use the kit to determine which progeny of the cross contains the desired genetic alteration. Thus, methods of using this kit are also included. The complete set of assays for distinguishing the nine FAD2-1A mutants and the one FAD2-1B mutant described here are listed in Table 4. The description of the development and use of the assays are provided in Examples 2 and 3, infra.

After one obtains a genetically altered plant expressing the FAD2-1A/FAD2-1B phenotype, one can efficiently breed the genetically altered plant with other plants containing desired traits. One can use molecular markers (i.e., polynucleotide probes described below) based on the SNP of FAD2-1A and/or FAD2-1B genes to determine which offspring of crosses between the genetically altered plant and the other plant have the polynucleotide encoding FAD2-1A and/or FAD2-1B. This process is known as Marker Assisted Rapid Trait Introgression (MARTI). Briefly, MARTI involves (1) crossing the genetically altered FAD2-1A/FAD2-1B plant with a plant line having desired phenotype/genotype (“elite parent”) for introgression to obtain F1 offspring. The F1 generation is heterozygous for FAD2-1A and FAD2-1B genes. (2) Next, an F1 plant is be backcrossed to the elite parent, producing BC1F1 which genetically produces 50% wild-type and 50% heterozygote FAD2-1A/FAD2-1B plants. (3) PCR using the polynucleotide probes is performed to select the heterozygote genetically altered plants containing FAD2-1A and FAD2-1B genes. (4) Selected heterozygotes are then backcrossed to the elite parent to perform further introgression. (5) This process of MARTI is performed for another four cycles. (6) Next, the heterozygote genetically altered plant is self-pollinated to produce BC6F2 generation. The BC6F2 generation produces a phenotypic segregation ratio of 3 wild-type parent plants to 1 genetically altered FAD2-1A/FAD2-1B plant. (7) One selects genetically altered FAD2-1A/FAD2-1B plants at the BC6F2 generation at the seedling stage using PCR with the polynucleotide probes and can optionally be combined with phenotypic selection at maturity. These cycles of crossing and selection can be achieved in a span of 2 to 2.5 years (depending on the plant), as compared to many more years for conventional backcrossing introgression method. Thus, the application of MARTI using PCR with polynucleotide probes significantly reduces the time to introgress the FAD2-1A/FAD2-1B genetic alterations into elite lines for producing commercial hybrids. The final product is an inbred plant line almost identical (99%) to the original elite in-bred parent plant that is the homozygous for FAD2-1A and FAD2-1B genes.

This invention utilizes routine techniques in the field of molecular biology. Basic texts disclosing the general methods of use in this invention include Sambrook et al., Molecular Cloning—A Laboratory Manual (2nd Ed.), Vol. 1-3, Cold Spring Harbor Laboratory, Cold Spring Harbor, N.Y. (1989); Kriegler, Gene Transfer and Expression: A Laboratory Manual (1990); and Ausubel et al. (eds.), Current Protocols in Molecular Biology (1994). Unless otherwise noted, technical terms are used according to conventional usage. Definitions of common terms in molecular biology maybe found in e.g., Benjamin Lewin, Genes IX, published by Oxford University Press (2007) (ISBN 0763740632); Krebs, et al. (eds.), The Encyclopedia of Molecular Biology, published by Blackwell Science Ltd. (1994) (ISBN 0-632-02182-9); and Robert A. Meyers (ed.), Molecular Biology and Biotechnology: a Comprehensive Desk Reference, published by VCH Publishers, Inc. (1995) (ISBN 1-56081-569-8).

The terms “approximately” and “about” refer to a quantity, level, value or amount that varies by as much as 30% in one embodiment, or in another embodiment by as much as 20%, and in a third embodiment by as much as 10% to a reference quantity, level, value or amount. As used herein, the singular form “a”, “an”, and “the” include plural references unless the context clearly dictates otherwise. For example, the term “a bacterium” includes both a single bacterium and a plurality of bacteria.

Having now generally described this invention, the same will be better understood by reference to certain specific examples and the accompanying drawings, which are included herein only to further illustrate the invention and are not intended to limit the scope of the invention as defined by the claims. The examples and drawings describe at least one, but not all embodiments, of the inventions claimed. Indeed, these inventions may be embodied in many different forms and should not be construed as limited to the embodiments set forth herein; rather, these embodiments are provided so that this disclosure will satisfy applicable legal requirements.

### Example 1 Generation of Genetically Altered Soybean Plants Producing Elevated Levels of Oleic Acid

Genetically altered soybean plants were generated by treating soybean line Williams-82 seeds (wild-type) with N-nitroso-N-methylurea (NMU) and initial genetically altered soybean plants were isolated using the protocols described in Ritchie, R., et al., 2004, Targeting Induced Local Lesions in Genomes. In Legume Crop Genomics, Wilson, et al., eds., pp 194-203 AOCS Press, Champaign, Ill. USA. All genetically altered soybean lines were grown in the field in West Lafayette, Ind. Five-seed M3 bulks from over 5,000 altered soybean lines were screened by gas chromatography (GC) to identify lines with elevated and reproducible levels of oleic acid. The protocol for gas chromatography of soybean seed samples to determine the relative levels of the five major soybean fatty acids (palmitic acid, stearic acid, oleic acid, linoleic acid, and linolenic acid) is described in Thapa, R., et al., 2016, Crop Science doi:10.2135/cropsci2015.09.0597 and references therein. Reproducibility was determined by GC analysis of five-seed bulks from individual M4 plants in the subsequent growing season. A two-tailed t-test analysis was performed to assess significance of difference between soybean line Williams-82 wild-type seed and each genetically altered soybean line for each fatty acid. From the screen, sixteen soybean lines were identified with reproducible elevated levels of oleic acid as described in Hudson, K., 2012, Intern. J. Agronomy 2012:7.doi:10.1155/2012/569817.

Nine genetically altered soybean lines with elevated oleic acid were further investigated. Because M₃ seed may possibly have been descended from heterozygous M₂ plants, M₄ seed from M₃ individuals were rescreened for homozygosity or homogeneity of oleic acid levels using gas chromatography, and, in all but three cases, it was determined that the original M₂ isolates were heterozygous. Fatty acid composition as a percentage of total fatty acids present from homozygous M₄ lines is shown in Table 3, infra. Upon regrowth of homozygous M₄ genetically altered individuals in the field in the 2014 growing season, genetically altered lines contained from 27% to 40% oleic acid in the oil fraction (fatty acid content) in the seeds. In contrast, wild-type Williams-82 soybean seed (from the same growing season) contained 21% oleic acid. All of these genetically altered soybeans showed a significant increase in oleic acid levels and most showed a statistically significant reduction in linoleic acid levels.

TABLE 3

| | | Palmitic Acid | Stearic Acid | Oleic Acid | Linoleic Acid | Linolenic Acid |
|---|---|---|---|---|---|---|
| ID^{†} | Mutation | (%) | (%) | (%) | (%) | (%) |
| W82 | N/A | 11.25 ± 0.67 | 3.58 ± 0.48 | 20.69 ± 1.83 | 56.26 ± 1.58 | 8.21 ± 00.85 |
| 1 | FAD2-1A_{L41F} | 10.17 ± 0.33 | 3.78 ± 0.13 | 29.71 ± 1.52*** | 49.86 ± 1.11* | 6.47 ± 0.19* |
| 2 | FAD2-1A_{V106M} | 9.46 ± 0.28*** | 3.55 ± 0.17 | 40.54 ± 1.99*** | 39.54 ± 1.53*** | 6.91 ± 0.38* |
| 3 | FAD2-1A_{S154F} | 9.63 ± 0.25* | 3.87 ± 0.21 | 31.87 ± 1.55*** | 47.64 ± 1.23*** | 6.99 ± 0.39* |
| 4 | FAD2-1A_{P163S} | 10.15 ± 0.53 | 3.49 ± 0.21 | 31.38 ± 4.02* | 50.27 ± 3.45* | 4.76 ± 0.24*** |
| 5 | FAD2-1A_{W194STOP} | 8.64 ± 0.10*** | 3.36 ± 0.18 | 39.07 ± 0.78*** | 41.66 ± 0.91*** | 7.28 ± 0.13 |
| 6 | FAD2-1A_{G204D} | 9.58 ± 0.11*** | 3.86 ± 0.17 | 35.85 ± 0.38*** | 43.23 ± 0.29*** | 7.47 ± 0.22* |
| 7a | FAD2-1A_{P284L} | 9.87 ± 0.11* | 3.17 ± 0.14 | 36.89 ± 2.17*** | 43.01 ± 1.79*** | 7.06 ± 0.40 |
| 7b | FAD2-1A_{P284S} | 9.94 ± 0.16 | 4.03 ± 0.06 | 30.30 ± 2.31* | 49.52 ± 2.07* | 6.11 ± 0.35* |
| 8 | FAD2-1A_{A358T} | 9.71 ± 0.27* | 4.08 ± 0.05 | 27.27 ± 2.42* | 58.87 ± 2.19 | 6.07 ± 0.46* |

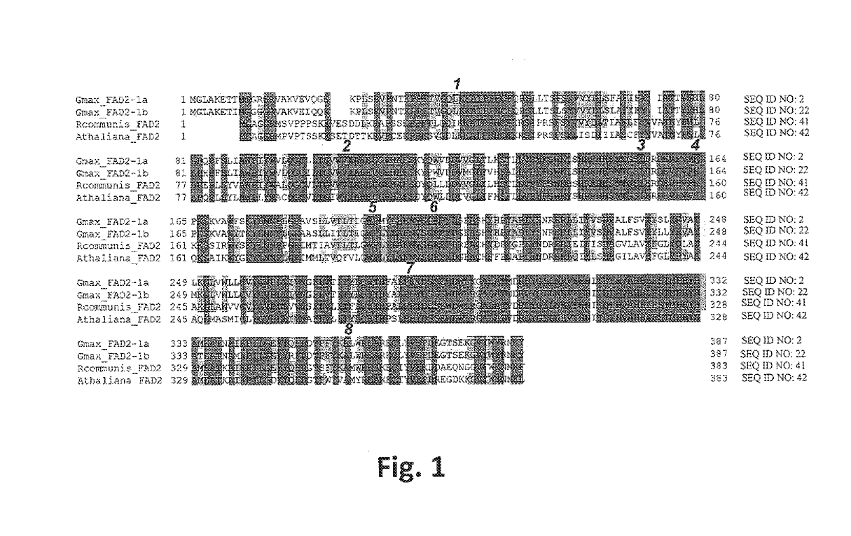

^{†}The left column (ID) refers to the corresponding numbers in FIG. 1 indicating the location of the mutations.
*Significant at the 0.05 probability level.
***Significant at the 0.001 probability level.

### Example 2 Identification of Genetic Mutations of Soybean Lines with Elevated Oleic Acid Levels

To determine if the elevated oleic acid phenotype in the mutant lines was caused by polymorphisms in the FAD2-1A gene, FAD2-1A (Glyma10g42470/Glyma.10G278000) was amplified and sequenced from the nine genetically altered soybean lines listed in Table 3, supra.

Plant DNA for sequencing was prepared as described in Carrero-Colon, et al., 2014, PLoSOne 9:e97891. The FAD2-1A gene was amplified from each mutant using the following FAD2-1A paired forward and reverse amplification primers: Primer KK 317 (forward) 5′-TGAGGGATTGTAGTTCTGTTGG-3′ (SEQ ID NO: 25); Primer KK 318 (reverse) 5′-AGCGTGCATTTTAGGCAGAA-3′ (SEQ ID NO: 26); Primer MCC 34 (mid-section forward) 5′-TGGCCAAAGTGGAAGTTCAA-3′ (SEQ ID NO: 27); and Primer MCC 35 (mid-section reverse) 5′-ATTGGTTGCTCCATCAATACTTGT-3′ (SEQ ID NO: 28). Multiple dye-terminator sequencing reactions were performed with the Big Dye Direct Cycle Sequencing Kit (Life Technologies, Grand Island, N.Y.) following manufacturer's recommended protocols using these amplification products as templates with the four primers described supra to obtain thorough two-stranded coverage of the full coding region of the FAD2-1A gene for each sample. Sequencing was performed at the Purdue University Genomics Core Facility. DNA for genotyping F2 seeds was prepared using the E-Z 96 Plant DNA Kit (Omega Bio-Tek, Norcross, Ga.) following manufacturer's recommended protocol using a seed chip <10% of the total seed volume pulverized in a GenoGrinder 2000 (SPEX Sample Prep, Metuchen, N.J.).

Each genetically altered soybean line carried a distinct single nucleotide polymorphism in the coding sequence of the FAD2-1A gene. In seven instances, the mutation resulted in a missense mutation in a highly conserved residue. See FIG. 1. In one instance the polymorphism resulted in a nonsense mutation at position 194. Two of the mutants possessed distinct alterations at one amino acid site, proline 284, which in one soybean line was changed to serine and in another soybean line was changed to leucine. No three-dimensional structure is available for the FAD2 enzyme. Yet, FAD2-1A_{V106M}, a missense mutation, occurs in a proposed transmembrane domain. The other mutations occur in portions of the polypeptide that are proposed to be located in the cytoplasmic domain of the enzyme which includes the catalytic site (Dyer and Mullen, 2001, FEBS Letters 494:44-47; Tang, et al., 2005, The Plant J. 44:433-446.

The SNPs for FAD2-1A_{V106M}, FAD2-1A_{G204D}, FAD2-1A_{W194STOP}, FAD2-1A_{P163S} and FAD2-1A_{P284S} all introduced a change in a restriction site. To verify that these new genetic alterations are associated with the observed elevation of oleic acid levels, co-dominant Cleaved Amplified Polymorphic Sequence (CAPS) markers were designed to detect the five single base polymorphisms (SNPs) within segregating populations using the protocol set forth in Neff, et al., 2002 (Trends in Genetics 18:613-615). The primers and enzyme combination for each of these alleles are described in Table 4, infra.

The polymorphisms in the FAD2-1A_{A358T}, FAD2-1A_{P284L} and FAD2-1A_{S154F} genetically altered soybeans did not introduce restriction sites, therefore derived Cleaved Amplified Polymorphic Sequence (dCAPS) markers are generated using the protocol set forth in Head, et al., 2012 (Mol. Breeding 30:1519-1523). Restriction enzymes (New England Biosciences) for genotyping were used according to manufacturer's recommended protocols. Briefly, after amplification in 20 μL reactions, 10 μL of each PCR reaction was digested overnight in a 40 μL digestion volume and electrophoresed on an agarose gel. See Table 4, infra. Mutant gene sequences are deposited into GenBank with the accession numbers KM594251-KM594258 but have not been publicly available prior to the filing date of this patent application.

TABLE 4

| | | | Amplicon | | |
|---|---|---|---|---|---|
| Line/ | | | size | PCR | |
| Mutation | Primer | Sequence(5′-3′) | (bp) | program* | Enzyme |
| 17015/FAD2 | KK 711 | TGATGACACACCATTTTACCAG (SEQ ID NO: 29) | 186 | 3 | BstNI (dCAPS) |
| 1A_A358T | KK 712 | CATTCTACTAATTATGTACTAATACATGAC | | | Cuts wild type |
| | | (SEQ ID NO: 30) | | | |
| 17560/FAD2 | KK 721 | TCCCATTCTGATGAATCGTCCTGA (SEQ ID NO: 31) | 178 | 2 | Bsu36I (dCAPS) |
| 1A_P284L | KK 722 | TGATGTTGCTTTGTTTTCTGTG (SEQ ID NO: 32) | | | Cuts wild type |
| 17451/FAD2 | KK 317 | TGAGGGATTGTAGTTCTGTTGG (SEQ ID NO: 25) | 1135 | 1 | BsmAI (CAPS) |
| 1A_P284S | MCC 35 | ATTGGTTGCTCCATCAATACTTGT (SEQ ID NO: 28) | | | Cuts mutant |
| 19296/FAD2 | KK 317 | TGAGGGATTGTAGTTCTGTTGG (SEQ ID NO: 25) | 1259 | 1 | BsmAI (CAPS) |
| 1A_P163S | MCC 35 | ATTGGTTGCTCCATCAATACTTGT (SEQ ID NO: 28) | | | Cuts mutant |
| 14184/FAD2 | KK 317 | TGAGGGATTGTAGTTCTGTTGG (SEQ ID NO: 25) | 1259 | 1 | BstEII (CAPS) |
| 1A_W194Stop | MCC 35 | ATTGGTTGCTCCATCAATACTTGT (SEQ ID NO: 28) | | | Cuts mutant |
| 14752/FAD2 | MCC 34 | TGGCCAAAGTGGAAGTTCAA (SEQ ID NO: 27) | 1135 | 1 | AccI (CAPS) |
| 1A_G204D | MCC 35 | ATTGGTTGCTCCATCAATACTTGT (SEQ ID NO: 28) | | | Cuts wild type |
| 17203/FAD2 | MCC 34 | TGGCCAAAGTGGAAGTTCAA (SEQ ID NO: 27) | 1135 | 1 | FokI (CAPS) |
| 1A_V106M | MCC 35 | ATTGGTTGCTCCATCAATACTTGT (SEQ ID NO: 28) | | | Cuts mutant |
| 17553/FAD2 | KK 735 | CCATCACTCCAACACAGGAT (SEQ ID NO: 33) | 153 | 2 | BamHI(dCAPS) |
| 1A_S154F | KK 736 | CATAGGCCACCCTATTGTGAG (SEQ ID NO: 34) | | | Cuts mutant |
| 19372/FAD2 | KK 34 | CCATGAAGCAGTTGCTGAAGCTGAT | 500 | 1 | BpuEI (CAPS) |
| 1A_L41F | KK 710 | (SEQ ID NO: 35) | | | Cuts wild type |
| | | CCTAGAGGGTTGTTTAAGTACTTGGAAA | | | |
| | | (SEQ ID NO: 36) | | | |
| 14473/FAD2- | KK315 | TCAGCAACAACAACTGAACTGAA (SEQ ID NO: 37) | 1132 | 1 | BsmAI (CAPS) |
| 1B_P284S | MCC37 | TGCTTGGTTCATCAATACTTGTT (SEQ ID NO: 40) | | | Cuts mutant |

*PCR Programs:
1. 95° C. 60 s, 5x (94° C. 30 s, 54° C. 20 s, 68° C. 4 m), 25x (94° C. 30 s, 56° C. 20 s, 68° C. 4 m), 68° C. 10 m, 4° C. soak.
2. 95° C. 60 s, 7x (94° C. 30 s, 56° C. 30 s, 68° C. 1 m), 28x (94° C. 30 s, 58° C. 20 s, 68° C. 1 m), 68° C. 4 m, 4° C. soak.
3. 95° C. 60 s, 7x (94° C. 30 s, 52° C. 30 s, 68° C. 1 m), 28x (94° C. 30 s, 54° C. 20 s, 68° C. 1 m), 68° C. 4 m, 4° C. soak.

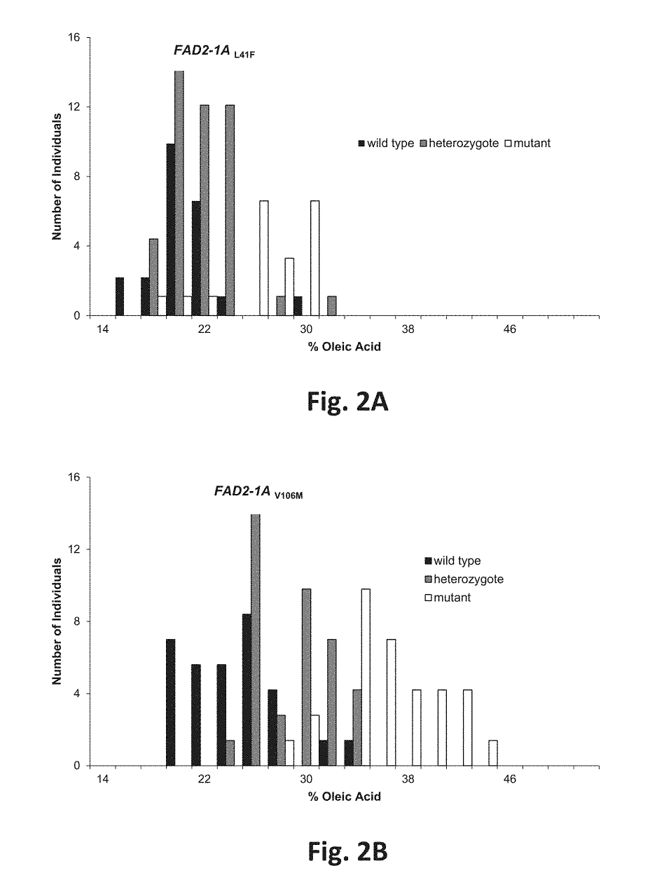

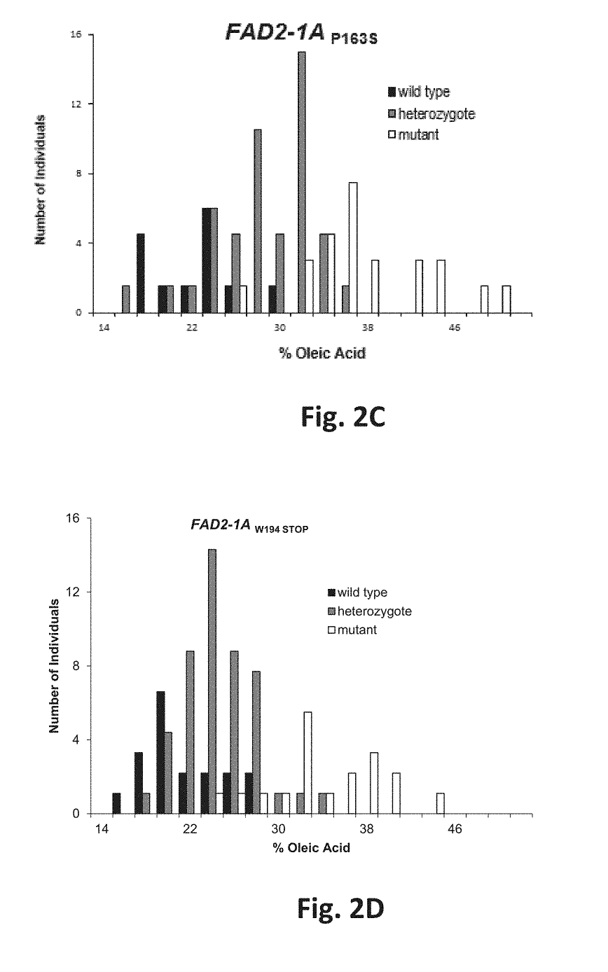

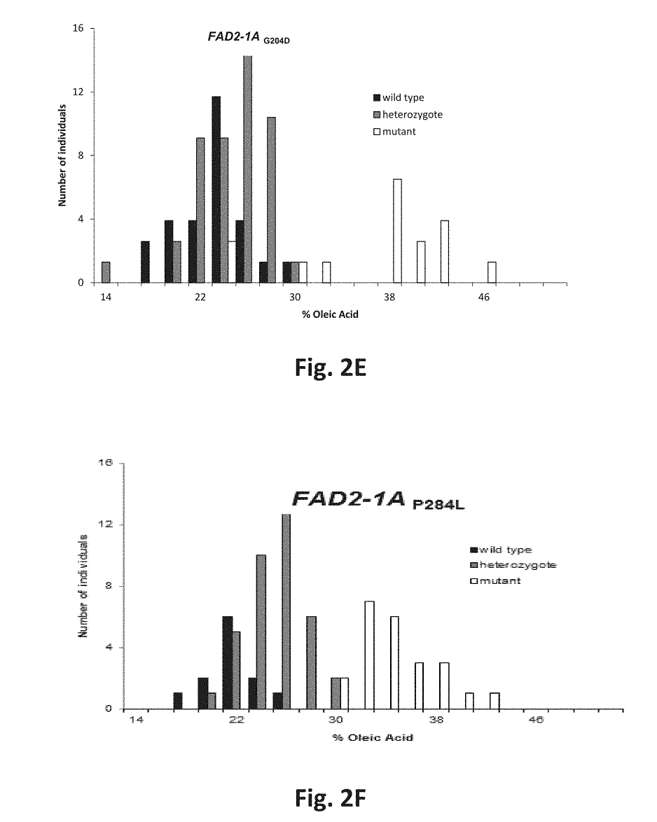

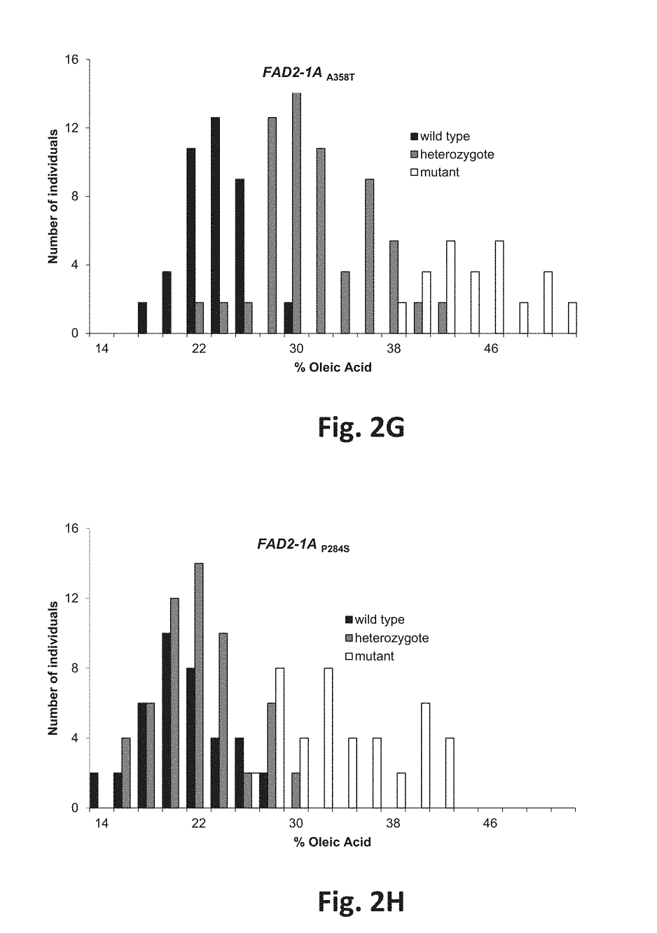

For FAD2-1A_{V106M}, bulked F₃ seed produced from genotyped BC₁ F₂ individuals were assayed for oleic acid content and for FAD2-1A_{L41F} and FAD2-1A_{W194STOP}, individual BC₁ F₂ seeds were chipped and genotyped (Table 5, infra). The protocol for the analysis of seed chips by GC is provided in Thapa, R., et al., 2016, Crop Science doi:10.2135/cropsci2015.09.0597 and the protocol for the preparation of nucleic acids from seed chips is described in Thapa, et al., 2016, Crop Science 56:226-231. For the remaining lines, the segregating M₃ seed from heterozygous M₂ plants were phenotyped using gas chromatography on chips from individual seeds using the protocol described supra, and DNA extracted from the remainder of the seed was genotyped using the protocol described supra. FIG. 2 shows the number of individuals in each bin plotted against the percentage oleic acid content in seed oil for homozygous genetically altered, heterozygous, and wild type individuals. Note that the novel fad2-1a mutation and the elevated oleic acid phenotype cosegregate in each population. The association of genotype and elevated oleic acid phenotype is consistent within each population, although the seed source and growth season varied.

TABLE 5

| | Wild type (+/+) | (n) | Heterozygote (+/m) | (n) | Mutant (m/m) | (n) |
|---|---|---|---|---|---|---|
| FAD2-1A_{L41F} | 21.70 ± 2.74 | 21 | 22.98 ± 2.66 | 41 | 27.60 ± 3.42*** | 18 |
| FAD2-1A_{V106M} | 25.61 ± 3.95 | 24 | 30.11 ± 3.02*** | 28 | 37.57 ± 4.04*** | 25 |
| FAD2-1A_{P163S} | 23.45 ± 3.38 | 11 | 29.60 ± 4.28*** | 34 | 38.83 ± 6.05*** | 19 |
| FAD2-1A_{W194STOP} | 22.78 ± 3.67 | 18 | 25.70 ± 3.1* | 44 | 35.13 ± 5.13*** | 18 |
| FAD2-1A_{G204D} | 24.02 ± 3.04 | 22 | 25.64 ± 3.01 | 37 | 37.99 ± 6.43*** | 15 |
| FAD2-1A_{P284L} | 23.12 ± 2.10 | 12 | 26.17 ± 2.11*** | 38 | 35.19 ± 3.18*** | 23 |
| FAD2-1A_{P284S} | 21.93 ± 3.75 | 19 | 23.08 ± 3.78 | 28 | 34.87 ± 5.14*** | 21 |
| FAD2-1A_{A358T} | 28.53 ± 2.75 | 22 | 32.67 ± 4.44*** | 36 | 45.67 ± 3.94*** | 15 |

*Significant at the 0.05 probability level.
***Significant at the 0.001 probability level.

Based on data presented supra, stronger FAD2-1A mutant alleles, such as FAD2-1A_{A358T} and FAD2-1A_{P163S}, appear to exhibit some degree of semi-dominance for the elevated oleic acid trait, and the weak alleles, FAD2-1A_{G204D} and FAD2-1A_{P284S}, appear recessive (see Table 5 supra). Not wishing to be bound to any particular hypothesis, it is possible that the subtle increase in oleic acid observed in individuals heterozygous for FAD2-1A_{P163S} or FAD2-1A_{A358T} may result from haploid insufficiency or potentially from the formation of incompletely functional dimers. Further, the extent of the oleic acid phenotype observed in heterozygotes does not appear to be consistent with subunit poisoning.

These new alleles provide a range of phenotypic severity for increased oleic acid level (30-40%) in soybean seeds. Some of these non-transgenic alterations, in combination with other genes affecting oleic and linolenic acid content, may be used in conventional breeding approaches for an improved soybean crop.

### Example 3 Identification of Genetically Modified Soybean Line Having Alteration in FAD2-1B

In screening the genetically altered soybean lines generated via NMU mutagenesis in Example 1 supra, seven lines with elevated oleic acid levels did not have a mutation in FAD2-1A. To learn the genetic alternation that generated the higher oleic acid levels in seeds, the coding region of the FAD2-1B gene was amplified and sequenced. DNA for sequencing was prepared as described in Carrero-Colón, et al., 2014. The FAD2-1B gene was amplified with primers KK 315 (forward) 5′-TCAGCAACAACAACTGAACTGAA-3′ (SEQ ID NO: 37) and KK 316 (reverse) 5′-TCGCTACAAGCTGTTTCACAAT-3′ (SEQ ID NO: 38) using PCR conditions described in Table 4 (PCR program 1), supra. The isolated amplicon was sequenced using the BigDye Direct Cycle Sequencing Kit (Life Technologies, Grand Island, N.Y.) using the amplified PCR products as templates with the amplification primers as well as two internal primers MCC 36 (forward) (5′-GTGGCCAAAGTTGAAATTCAG-3′) (SEQ ID NO: 39) and MCC 37 (reverse) (5′-TGCTTGGTTCATCAATACTTGTT-3′) (SEQ ID NO: 40) for complete two-stranded coverage. Sequencing of PCR products was performed at the Purdue University Genomics Core Facility.

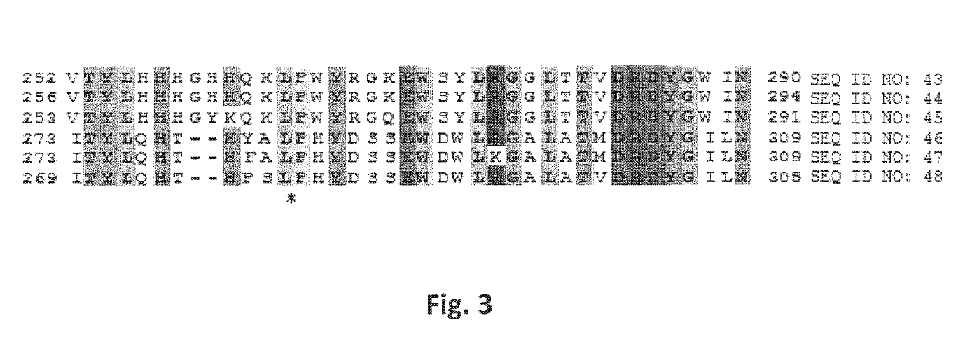

Six lines do not carry a polymorphism in the coding region of FAD2-1A or FAD2-1B. See Hudson, 2012. These six lines are being investigated for the cause of the high oleic phenotype. In one line (line 14473), however, a single nucleotide polymorphism was identified that caused a missense mutation in the coding region of FAD2-1B. This line carried a C to T transition mutation resulting in a substitution of serine for proline at position 284 (P284S) in the amino acid sequence of the predicted protein. See FIG. 3 for the location of the mutation and its relationship to other FAD sequences. SEQ ID NO: 24 contains the full-length amino acid sequence of this altered FAD2-1B. SEQ ID NO: 23 is the cDNA sequence for this P284S mutation in FAD2-1B. Interestingly, a mutation resulting in the same substitution at this position was identified in the FAD2-1A gene supra; see SEQ ID NO: 17 and 18 and FIG. 3.

Seeds from this genetically altered soybean plant produced in the field in 2014 contained 34% oleic acid in the oil fraction, in contrast to the Williams-82 wild type soybean plants that contained 22% oleic acid, a significant increase in oleic acid. See Table 6.

TABLE 6

| | Palmitic acid | Stearic acid | Oleic acid | Linoleic acid | Linolenic acid |
|---|---|---|---|---|---|
| | (%) | (%) | (%) | (%) | (%) |
| Williams82 | 10.93 ± 0.75 | 3.69 ± 0.35 | 22.52 ± 1.43 | 54.72 ± 1.53 | 8.14 ± 0.46 |
| Line 14473 | 11.3 ± 0.21^{NS} | 4.8 ± 0.48*** | 34.5 ± 1.33*** | 43.7 ± 0.89*** | 5.6 ± 0.32*** |

***Significant at the 0.001 probability level.
^{NS}Not significant

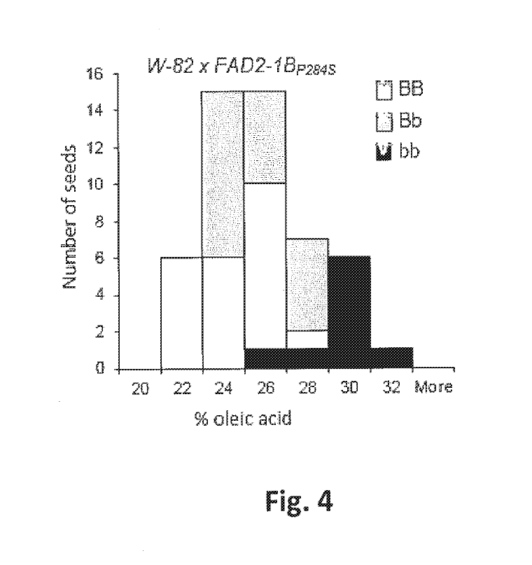

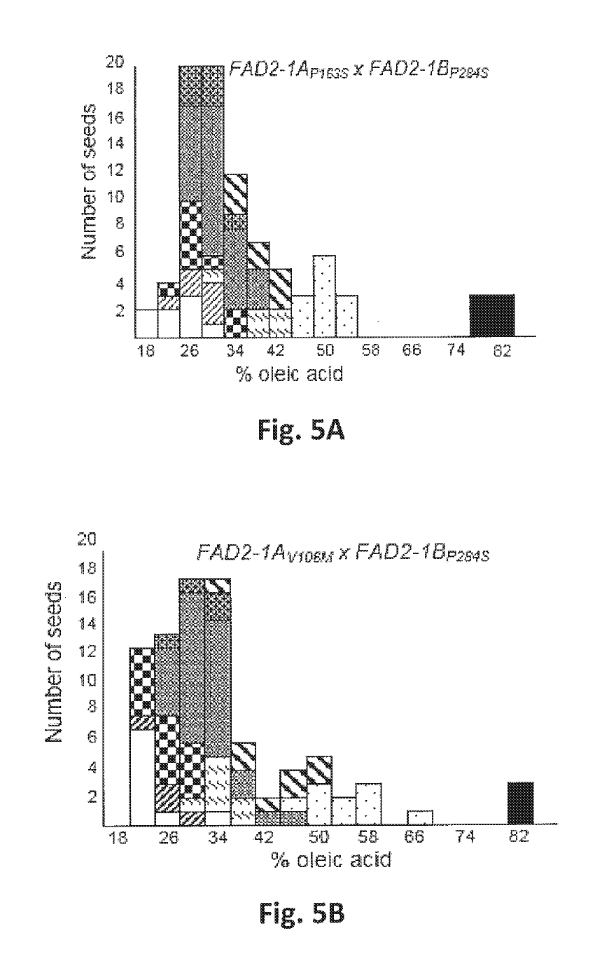

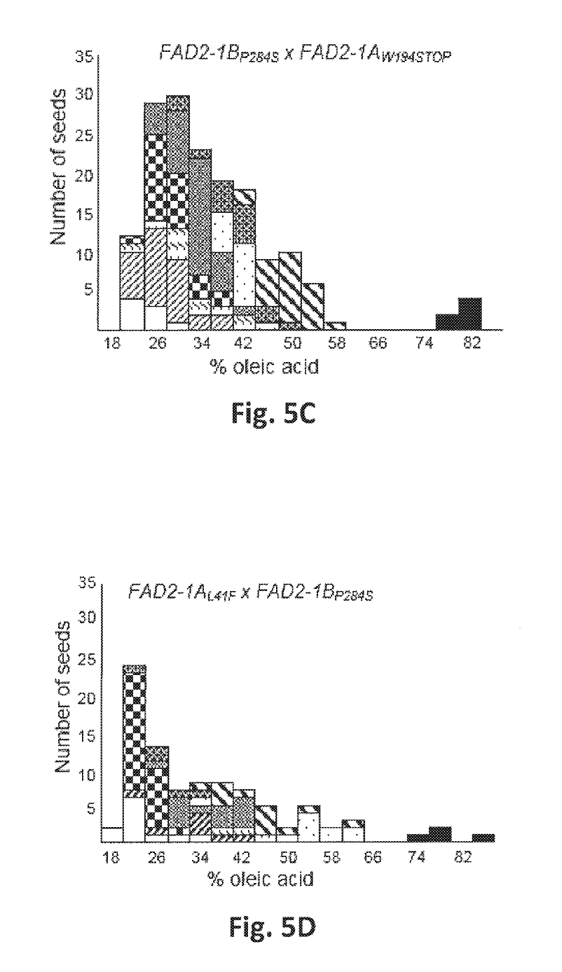

To determine if the increased levels of oleic acid in the mutant was linked to the newly identified mutation in the FAD2-1B coding region, the P284S mutant soybean plant was crossed to the Williams-82 wild type parent (BC₁). The F₁ plant was allowed to self-pollinate in the field and segregating F₂ seeds were chipped for fatty acid content analysis and genotyped for the FAD2-1B_{P284S} polymorphism. DNA from seed chips was prepared and genotyping for FAD2-1A was performed using the procedures and markers described supra. The single base change in FAD2-1B_{P284S} introduced a BsmAI restriction site, so primers KK315 and MCC37 were used to amplify a fragment of the FAD2-1B gene in wild-type, heterozygote and mutant alleles soybean plants using the protocol described supra. The PCR fragments were then subjected to BsmAI (New England Biolabs, Ipswich, Mass.) digestion using the manufacturer's recommended protocol. FIG. 4 shows clear co-segregation of the elevated oleic acid levels with the mutant form of the FAD2-1B gene. This polymorphism thus provides a CAPS (Cleaved Amplified Polymorphic Sequence) molecular marker for the causative allele (Konieczny and Ausubel, 1993).

### Example 4. Oleic Acid Levels in a Double Mutant Soybean Plant

It has been previously shown that mutations in the FAD2-1A and FAD2-1B genes have a synergistic effect on seed oleic acid content, resulting in plants with a higher seed oleic acid content that either parent. See, e.g., Hoshino, et al., 2010; Pham, et al., 2010. To determine the effect of combining this novel allele of FAD2-1B_{P284S} with a variety of the FAD2-1A mutant alleles discussed supra, the FAD2-1B_{P284S} line was crossed to four alleles of fad2-1a, including FAD2-1A_{L41F}, FAD2-1A_{V106M}, FAD2-1A_{W194STOP}, and FAD2-1A_{P163S}. After harvesting, F₂ seeds were chipped for fatty acid profiling and genotyped for both the FAD2-1A and FAD2-1B mutations using the protocols discussed supra. In all four cases, a synergistic interaction was observed between the mutations in the fad2-1a/fad2-1b double mutants. On average, these double mutant soybean plants had approximately 78.4% oleic acid, a statistically significant increase. See Table 7, infra. Additionally, fad2-1a/fad2-1b double mutants had an average of 4.8% linolenic acid, a significant decrease from the Williams-82 wild type.

TABLE 7

| | FAD2-1A_{P163S} × | | FAD2-1A_{V106M} × | | FAD2-1A_{L41F} × | | FAD2-1B_{P284S} × | |
|---|---|---|---|---|---|---|---|---|
| | FAD2-1B_{P284S} | | FAD2-1B_{P284S} | | FAD2-1B_{P284S} | | FAD2-1A_{W194STOP} | |
| Genotype | % oleic acid | N | % oleic acid | N | % oleic acid | N | % oleic acid | N |
| AABB | 22.5 ± 3.3 | 8 | 21.6 ± 3.4 | 9 | 21.4 ± 4.4 | 11 | 23.0 ± 3.4 | 8 |
| AABb | 25.4 ± 3.3 | 6 | 24.3 ± 2.5 | 4 | 30.9 ± 6.6 | 7 | 26.0 ± 4.6 | 28 |
| AAbb | 37.2 ± 4.9 | 5 | 32.2 ± 3.3 | 7 | 39.7 ± 3.4 | 3 | 30.9 ± 6.4 | 11 |
| AaBB | 26.0 ± 3.4 | 9 | 23.9 ± 3.7 | 14 | 21.6 ± 2.2 | 25 | 27.3 ± 4.1 | 24 |
| AaBb | 29.5 ± 3.6 | 27 | 29.8 ± 4.6 | 30 | 33.4 ± 5.2 | 13 | 31.0 ± 4.1 | 33 |
| Aabb | 49.2 ± 3.0 | 12 | 52.8 ± 5.8 | 10 | 51.5 ± 7.3 | 10 | 38.7 ± 2.6 | 14 |
| aaBB | 28.2 ± 2.9 | 7 | 28.6 ± 4.4 | 4 | 26.2 ± 4.5 | 5 | 38.1 ± 5.4 | 15 |
| aaBb | 35.6 ± 3.1 | 8 | 40.9 ± 5.4 | 8 | 43.5 ± 7.7 | 12 | 47.8 ± 3.8 | 24 |
| aabb | 77.8 ± 2.1 | 6 | 81.2 ± 0.2 | 3 | 77.5 ± 4.0 | 4 | 78.3 ± 2.2 | 6 |
| Total | | 88 | | 89 | | 90 | | 163 |

This novel allele of FAD2-1B (namely, FAD2-1B_{P284S}) affects the paralogous, highly-conserved proline residue which is also mutated in FAD2-1A_{P284S} and FAD2-1A_{P284L}, which underscores the importance of this region of the desaturase molecule to enzyme function. Interestingly, of the allelic series of FAD2-1A mutations isolated supra, the P-284 mutations were not the most severe. Thus, the finding that when the FAD2-1B_{P284S} mutation is crossed with soybean plants having FAD2-1A_{L41F}, FAD2-1A_{V106M}, FAD2-1A_{W194STOP}, or FAD2-1A_{P163S} resulted in double mutants with such high oleic acid content. These double mutant plants have seeds that are competitive with commercial plants having high oleic traits. Soybean line 14473 (containing FAD2-1B_{P284S}) was crossed with pollen from soybean line 14184 (containing FAD2-1A_{W194STOP}) in the summer of 2013, the resulting F1 plant was grown in the field in West Lafayette in the summer of 2014, and seeds (F2) carrying both mutations were grown in the greenhouse over the winter of 2014/2015 and planted in the field in 2015 to generate bulk F4 seed. These F4 seed were deposited with ATCC and assigned accession number PTA-122890.

The foregoing detailed description and certain representative embodiments and details of the invention have been presented for purposes of illustration and description of the invention. It is not intended to be exhaustive or to limit the invention to the precise forms disclosed. It will be apparent to practitioners skilled in the art that modifications and variations may be made therein without departing from the scope of the invention. All references cited herein are incorporated by reference.

We, the inventors, claim as follows:

1. A genetically altered soybean plant and parts thereof comprising an altered delta-twelve fatty acid desaturase 2-1B enzyme (FAD2-1B) and an altered delta-twelve fatty acid desaturase 2-1A enzyme (FAD2-1A), wherein said altered FAD2-1B has reduced enzymatic activity compared to wild-type FAD2-1B, wherein said altered FAD2-1A has reduced enzymatic activity compared to wild-type FAD2-1A, and wherein the seeds of said genetically altered soybean plant contain higher amounts of oleic acid compared to the amount of oleic acid in the seeds of a wild-type soybean plant having wild-type FAD2-1B and wild-type FAD2-1A.

2. The genetically altered soybean plant of claim 1 wherein said altered FAD2-1B has a P284S mutation and the amino acid sequence of SEQ ID NO: 24.

3. The genetically altered soybean plant of claim 2 wherein said altered FAD2-1B is encoded by a polynucleotide having the sequence of SEQ ID NO: 23.

4. The genetically altered soybean plant of claim 2 wherein said altered FAD2-1A is a mutation selected from the group consisting of FAD2-1A_{L41F} having the amino acid sequence of SEQ ID NO: 4, FAD2-1A_{V106M} having the amino acid sequence of SEQ ID NO: 6, FAD2-1A_{S154F} having the amino acid sequence of SEQ ID NO: 8, FAD2-1A_{P163S} having the amino acid sequence of SEQ ID NO: 10, FAD2-1A_{W194STOP} having the amino acid sequence of SEQ ID NO: 12, FAD2-1A_{G204D} having the amino acid sequence of SEQ ID NO: 14, FAD2-1A_{P284L} having the amino acid sequence of SEQ ID NO: 16, FAD2-1A_{P284S} having the amino acid sequence of SEQ ID NO: 18, and FAD2-1A_{A358T} having the amino acid sequence of SEQ ID NO: 20.

5. The genetically altered soybean plant of claim 4, wherein said FAD2-1A_{L41F} is encoded by a polynucleotide having the sequence of SEQ ID NO: 3; wherein said FAD2-1A_{V106M} is encoded by a polynucleotide having the sequence of SEQ ID NO: 5; wherein said FAD2-1A_{S154F} is encoded by a polynucleotide having the sequence of SEQ ID NO: 7; wherein said FAD2-1A_{P163S} is encoded by a polynucleotide having the sequence of SEQ ID NO: 9; wherein said FAD2-1A_{W194STOP} is encoded by a polynucleotide having the sequence of SEQ ID NO: 11; wherein said FAD2-1A_{G204D} is encoded by a polynucleotide having the sequence of SEQ ID NO: 13; wherein said FAD2-1A_{P284L} is encoded by a polynucleotide having the sequence of SEQ ID NO: 15; wherein said FAD2-1A_{P284S} is encoded by a polynucleotide having the sequence of SEQ ID NO: 17; and wherein said FAD2-1A_{A358T} is encoded by a polynucleotide having the sequence of SEQ ID NO: 19.

6. A seed of said genetically altered soybean plant of claim 4.

7. A cell of said genetically altered soybean plant of claim 4.

8. A protoplast of said cell of claim 7.

9. A germplasm of said genetically altered soybean plant of claim 4.

10. The genetically altered soybean plant of claim 4 wherein said altered FAD2-1B has a P284S mutation and wherein said altered FAD2-1A has a FAD2-1A_{W194STOP} mutation.

11. The genetically altered soybean plant of claim 10 having ATCC accession number PTA-122890.

12. A seed of said genetically altered soybean plant of claim 10.

13. A cell of said genetically altered soybean plant of claim 10.

14. A protoplast of said cell of claim 13.

15. A germplasm of said genetically altered soybean plant of claim 10.

16. A method for constructing a genetically altered soybean plant having an altered delta-twelve fatty acid desaturase 2-1B enzyme (FAD2-1B) and an altered delta twelve fatty acid desaturase 2-1A enzyme (FAD2-1A) and capable of producing seeds with a fatty acid content of at least 70% oleic acid, the method comprising:
(i) introducing a nucleic acid encoding an altered FAD2-1B enzyme having a P284S mutation into a wild-type soybean plant to provide a first genetically altered soybean plant;
(ii) selecting said first genetically altered soybean plant that is homozygous for said altered FAD2-1B enzyme,
(iii) introducing a nucleic acid encoding said altered FAD2-1A enzyme into a wild-type soybean plant to provide a second genetically altered soybean plant; wherein said altered FAD2-1A enzyme is selected from the group consisting of FAD2-1A_{L41F}, FAD2-1A_{V106M}, FAD2-1A_{S154F}, FAD2-1A_{P163S}, FAD2-1A_{W194STOP}, FAD2-1A_{G204D}, FAD2-1A_{P284L}, FAD2-1A_{P284S}, and FAD2-1A_{A358T}; and
(iv) selecting said second genetically altered soybean plant that is homozygous for said altered FAD2-1B enzyme and said altered FAD2-1A enzyme; thereby constructing said genetically altered soybean plant capable of producing seeds with a fatty acid content of at least 70% oleic acid.

17. The method of claim 16, wherein said FAD2-1B_{P284S} has the amino acid sequence of SEQ ID NO: 24; wherein said FAD2-1A_{L41F} has the amino acid sequence of SEQ ID NO: 4; wherein said FAD2-1A_{V106M} has the amino acid sequence of SEQ ID NO: 6; wherein said FAD2-1A_{S154F} has the amino acid sequence of SEQ ID NO: 8; wherein said FAD2-1A_{P163S} has the amino acid sequence of SEQ ID NO: 10; wherein said FAD2-1A_{W194STOP} has the amino acid sequence of SEQ ID NO: 12; wherein said FAD2-1A_{G204D} has the amino acid sequence of SEQ ID NO: 14; wherein said FAD2-1A_{P284L} has the amino acid sequence of SEQ ID NO: 16; wherein said FAD2-1A_{P284S} has the amino acid sequence of SEQ ID NO: 18; and wherein said FAD2-1A_{A358T} has the amino acid sequence of SEQ ID NO: 20.

18. The method of claim 16, wherein said introducing said at least one nucleic acid encoding either said altered FAD2-1A or FAD2-1B occurs via introgression, genomic editing, or exposing said wild-type soybean plant to a mutagen, and said selecting said genetically altered soybean plant occurs via marker assisted selection.

19. A genetically altered soybean plant produced using the method of claim 16.

20. The seeds of said genetically altered soybean plant of claim 19.

21. A kit useful for the identification of SNPs in FAD2-1A or FAD2-1B genes of a soybean plant comprising a plurality of polynucleotides, optionally a restriction endonuclease, optionally a polymerase, optionally an identifying dye, and optionally instructions; wherein said plurality of polynucleotides are selected from group consisting of SEQ ID NOs: 29 and 30, SEQ ID NOs: 31 and 32, SEQ ID NOs: 25 and 28, SEQ ID NOs: 27 and 28, SEQ ID NOs: 33 and 34, SEQ ID NOs: 35 and 36, SEQ ID NOs: 37 and 40, and a combination thereof.

22. The kit of claim 21, wherein said optional endonuclease is BstNI for polynucleotides having SEQ ID NOs: 29 and 30; wherein said optional endonuclease is Bsu36I for polynucleotides having SEQ ID NOs: 31 and 32; wherein said optional endonuclease is selected from the group consisting of BsmAI and BstEII for polynucleotides having SEQ ID NOs: 25 and 28; wherein said optional endonuclease is selected from the group consisting of Accl and Fokl for polynucleotides having SEQ ID NOs: 27 and 28; wherein said optional endonuclease is BamHI for polynucleotides having SEQ ID NOs: 33 and 34; wherein said optional endonuclease is BpuEI for polynucleotides having SEQ ID NOs: 35 and 36; and wherein said optional endonuclease is BsmAI for polynucleotides having SEQ ID NOs: 37 and 40.
